SEQUENCE LISTING (25 sequences)

SEQ ID NO: 1 - DNA, 1978 bp, synthetic construct
  source 1..1978: mol_type = other DNA
aggagttgtgttttttaagaaagaagattttagctgggcatggtggcacacgcctttaatcctagcacttgagaggcagaggcaggcaaatttctgagttcgaggccagcatggtctagaaagtgagtgccaggacagccagggctatacagagaaaccctgtctcaaaaaaccaaaaaaaaaaaaaaaaaaaaaaaaaaaaagactttaagtatcaattagaattagactttttcctcactttatttactcatgaaattggctgaaaagtaatagaaatggaaaaacaaaaacaaaaaacaagttgcggaggcttctgaaatactaactagaccaggagtgagatgatcttgggcagagggcagaagaacatggtatccaagagattctgaggccttgtctataaagtctgagcgccactggtctttctttttaaagttctctttcaaccttatagtgaggaaattcatctttcttaaaacttgtctgtatttatttatccattagcataactttagagtatatggattcaacctgactttattattatttagcatattctttaatgtagagatggtagatttttttttttttatttaggaaatcggctagcttttcgttaatttatttacattttcttttttttttatttgcatgaggtggctagttaactttatacagtatagatattttaatgctcttcttgctctaatgtaatgggttcattaaaaatgtgtacaatctagagtaaaaacaaccaaaggcctaggagaaagcaagaaaagaggcactgagaatgctgatttttttttttctataaacattatgtgggaaaaaggaagggatgctgatagttaacacacaagttgtttctggtgtctcaggggcagtcagctggcactcaaatgttctcttccagtgctggttaaccggcacctccttggctggactcctcaacatccttgtgtttagggagaaagaagtctgattggtgtcatggatgattaaccgctaatttagctcccaagtgtgaatgaaaaagggtactaagatctcagaggtgatgtgagatacaggaggggtcacgctgttttaatttagtgcacggttgaactcaaaaatttggatcagatagaaagaaatgtttctccctccttttcgacccccacccccacccccatttctttcctctgtttgcaaagctctcagtgcccttgcccttaagaacagcgaagagtcatttgacacgtctgaaagctggaggcgagttttacttttcaggtcatccaatttaggagatccaatttctgcggaggggagaaaaaaactgggtgggggagggaaacagcagactcctggtcttccgatctgtctaccttcaatacacaacctgacatgcagatccagccaagtcagggcttttacttgaacctccactaccagccccagcagcagcagcacaaccttgtcactcattccagagaaacacgccccattcctcttgaccaataatggctccactgcctgcatagtaatgagctcgagacctcccctgaccaatagcgctcccggagcggggttagttttgcatgtacctaaatgatttgcataacccggcggccaggggctcccgaggcgagcgtgcaaccctagaagggaaaaggacgcgcggagcgggagccgcctcggggagagcgcggacaaccagggtgtttgtgagagctggggcgggggttgggacgcctggccggcatggctggaggctgcgctccgcaaccttgaggagctgtgcggctggaggaggcccgggacaggaggcggcggcgatggcagcgcgcggcccgggcagccgctctgggccgggtcggctggcctgagccgcggggctgccggtgcgcgtccatggagcagcgggaagggagaaactgcggagcgccgcgtcctaacgctccggcggcagactgctgaaggaagcgag

SEQ ID NO: 2 - DNA, 2940 bp, synthetic construct
  source 1..2940: mol_type = other DNA
gtagaaccgaagtccagcttatccatcagtcaacaagtcctgtgtgctctgcacaaagacaccaaggcaagtatcactacgatagggtactttcttcatggcccataagctccatgccacatatgcagaggggttgcagcccagttctattttttatctagcctgtggatgccgcaaatttacttacacacaaccaatgaaaactttaagaggaaccgatctgtagacagagaccctattattttttgcttgccgtcaatttttatttggtttatctcagtatctttccagtccttatgatttatttaagagctgatcatttttctctagttatctgagatattaccttaatcactggttttttgttttttgtctttatactttcttgggacatcttatccaagagatggagactaattgaccaatgtatctctaatgcaaagcatacaaaatgtaccaaataagaaatacaaacacttcaatatatcttatcttttctgttccctacccaagtcttttgttcctgatcttttatcttcttggctaagctgcaaatttgaatcttcctttcttacatttgtcatctaatagacaataggaatttattttttagtaaaatctgcatctccccttttttcaaaatataaatctctcctttcacctaagttttcagccataagattttattatttgaagacaaagaataaagttctccaagtgtggttcatgaatcacctgtgtcactttcacttgctgtgcaacaaaatgctggtttctgggccccacttaggcctactaaatcattctgcatttttaatcaagatttgcatataagccggactccgtggctcatgactgtaatcccagcactttgggaggccaaggcaggtggatcacttgaggtgaggagtttgaggccagcctggccaacatgagaaaccccatctctactaaaaaaaaaaaaaaaaaaaaaaaaaaaaaagtagctaaacatggtggtgggcacctgtaataccagctactggggaggctgaggcaggagaatcacttgaacccgggaggtggaggttgcagtgagccaagatcacgccactggacacagtgagtgagagtccgtttcaaaaaaaaaaaaaaaaaagaagagatcttaaagaaacgattacagaaactaactttacaatttcttccttatttgttcatgacagttgctagaaaataatggaaatgatttagacgatgaaagcctatttagggaaggctctaagagtaccagattaggagccaggagcccaggagctgttccgtgatgatgttgggcagaacacactctctctcgagcctcagatttcttatctataaaaccagagttttctgggggtctttaagttgtctttcagccctgcagtgagaggagatatgtcattcctaaaacttgaaatgaaggcatatgtttatttattaactctctagcttctagacagatttactgtatgtgacttatgtgcccaacctgacattatcttttagataaactcccaaatacaaagatgataaaattttttcattttaaacttataggtctgcatcaacctgtttccattgtcctaagttttatttacatacgatggctagtcaactttatatgataattatctttaaatcctgctttttttttttttttttttttttttttttgagacagagttttgctcttgttgcccaggctggaatgcaatggcgcgatctccagctcaccgcaacctccgcctcccgggctcaagcaattcgcctgcctcagcctcccgagtagctgggattacaggcatctgccaccacacccggctaattttgtatttttagtagagacggggtttcttcatgttggtcagggtggtctagaactcccaacctcaggtgatccgcccgccgcagcctcccaaagtgttgggattacaggcgtgagccaccgcgcccggccgaatcctgcttgttttaatgtattcattaaaaatgtctccattatagagtaaaacgactcaacgccatggcagagagcaagaaacgaagtacccttaaagccgtagttggtacaggtaatgattccgtggggtctattaggccacagggactcaaaatccagaagaggagggaatgctgataattacctaacaccaacgttgctgctggcttctcagccgtagtcacctcccactcagatgttttggaaattgtccagtgttcagaaacatcactttctctgaccttttacaaaattcctgtgtttcaggattttaaagtgcgtgagaaaggagtctaatttgcagtcaccaaaaacaccaaatacagagagaggactggggagaatgaattgctaagacttagctaccaaatgtgtatcaagataggtgtaaagatcccaggtgaagtgaggcactggagagggtacacttttaacttagtgtatagctaaaacccaaagatgtgtgtcagatcgagggaaacttttttcctcctcttcaaaatcttttttccgtttgcagagctccagacgtcctcgccctttggaacagcggagagtcatttaaaacttaactgaacgctggagggaagttctacttaccaggtcatccaatttaggagatccaatttctgcggagggaacaaaaagacgggtgggggagggaaaaactagaccccttgtcctactatccttctaccttcaatccacaacctgccagacaaatccagccaagtcaagattttgccttgaaccaccaccaccaccaccaccaccaccgccgccaccaccaccaccaccaccgccgccaccaccacaaccaccaccaccttgtcactcacgcccgagaaacacgcccctctggtcctcctcttggccaatggcggctctcctctctgcatagtaatgagctcgagacctcc

SEQ ID NO: 3 - DNA, 642 bp, synthetic construct
  source 1..642: mol_type = other DNA
ggagggaaacagcagactcctggtcttccgatctgtctaccttcaatacacaacctgacatgcagatccagccaagtcagggcttttacttgaacctccactaccagccccagcagcagcagcacaaccttgtcactcattccagagaaacacgccccattcctcttgaccaataatggctccactgcctgcatagtaatgagctcgagacctcccctgaccaatagcgctcccggagcggggttagttttgcatgtacctaaatgatttgcataacccggcggccaggggctcccgaggcgagcgtgcaaccctagaagggaaaaggacgcgcggagcgggagccgcctcggggagagcgcggacaaccagggtgtttgtgagagctggggcgggggttgggacgcctggccggcatggctggaggctgcgctccgcaaccttgaggagctgtgcggctggaggaggcccgggacaggaggcggcggcgatggcagcgcgcggcccgggcagccgctctgggccgggtcggctggcctgagccgcggggctgccggtgcgcgtccatggagcagcgggaagggagaaactgcggagcgccgcgtcctaacgctccggcggcagactgctgaaggaagcgag

SEQ ID NO: 4 - DNA, 20 bp, synthetic construct
  source 1..20: mol_type = other DNA
ggcagaggcaggcaaatttc

SEQ ID NO: 5 - DNA, 20 bp, synthetic construct
  source 1..20: mol_type = other DNA
ctcgcttccttcagcagtct

SEQ ID NO: 6 - DNA, 20 bp, synthetic construct
  source 1..20: mol_type = other DNA
ggagggaaacagcagactcc

SEQ ID NO: 7 - DNA, 20 bp, synthetic construct
  source 1..20: mol_type = other DNA
ctcgcttccttcagcagtct

SEQ ID NO: 8 - DNA, 20 bp, synthetic construct
  source 1..20: mol_type = other DNA
atggctggacacctggcttc

SEQ ID NO: 9 - DNA, 20 bp, synthetic construct
  source 1..20: mol_type = other DNA
tcagtttgaatgcatgggag

SEQ ID NO: 10 - DNA, 20 bp, synthetic construct
  source 1..20: mol_type = other DNA
atgtataacatgatggagac

SEQ ID NO: 11 - DNA, 20 bp, synthetic construct
  source 1..20: mol_type = other DNA
tgcccctgtcgcacatgtga

SEQ ID NO: 12 - DNA, 20 bp, synthetic construct
  source 1..20: mol_type = other DNA
atgcccctcaacgtgaactt

SEQ ID NO: 13 - DNA, 24 bp, synthetic construct
  source 1..24: mol_type = other DNA
ttatgcaccagagtttcgaagctg

SEQ ID NO: 14 - DNA, 20 bp, synthetic construct
  source 1..20: mol_type = other DNA
atgaggcagccacctggcga

SEQ ID NO: 15 - DNA, 22 bp, synthetic construct
  source 1..22: mol_type = other DNA
ttaaaagtgcctcttcatgtgt

SEQ ID NO: 16 - DNA, 20 bp, synthetic construct
  source 1..20: mol_type = other DNA
tccagggtttagggttggga

SEQ ID NO: 17 - DNA, 20 bp, synthetic construct
  source 1..20: mol_type = other DNA
ccttcctacaaacgcctcgt

SEQ ID NO: 18 - DNA, 20 bp, synthetic construct
  source 1..20: mol_type = other DNA
tttcgcaacgggtttgccgc

SEQ ID NO: 19 - DNA, 23 bp, synthetic construct
  source 1..23: mol_type = other DNA
aaggtcgcctctagctcctgtag

SEQ ID NO: 20 - DNA, 19 bp, synthetic construct
  source 1..19: mol_type = other DNA
ctccctccatgagggtctg

SEQ ID NO: 21 - DNA, 20 bp, synthetic construct
  source 1..20: mol_type = other DNA
tcttcggcttcgagggtaaa

SEQ ID NO: 22 - DNA, 22 bp, synthetic construct
  source 1..22: mol_type = other DNA
gcaaaggggactatgagaagga

SEQ ID NO: 23 - DNA, 19 bp, synthetic construct
  source 1..19: mol_type = other DNA
cgtttggggcgcttatggt

SEQ ID NO: 24 - DNA, 23 bp, synthetic construct
  source 1..23: mol_type = other DNA
gtagaaccgaagtccagcttatc

SEQ ID NO: 25 - DNA, 22 bp, synthetic construct
  source 1..22: mol_type = other DNA
ggaggtctcgagctcattacta

(19) United States
(12) Patent Application Publication
(10) Pub. No.: US 2026/0009014 A1
(43) Pub. Date: Jan. 8, 2026

(54) Electromagnetic Field-Responsive Gene Promoter and Use Thereof

(71) Applicant: DONGGUK UNIVERSITY INDUSTRY-ACADEMIC COOPERATION FOUNDATION, Seoul (KR)

(72) Inventors: Jongpil Kim, Seoul (KR); Junyeop Kim, Gyeonggi-do (KR)

(21) Appl. No.: 18/992,699

(22) PCT Filed: Aug. 10, 2023
(86) PCT No.: PCT/KR2023/011817

§ 371 Date: Jan. 9, 2025

(30) Foreign Application Priority Data
Aug. 10, 2022 (KR) 10-2022-0100060

(51) Int. Cl. C12N 13/00; C07K 14/705; C12N 15/85

(52) U.S. Cl. CPC C12N 13/00; C07K 14/705; C12N 15/85; C12N 2740/15043; C12N 2830/002

(57) ABSTRACT

The present invention relates to an Lgr4 gene promoter regulated by electromagnetic fields or a fragment thereof, a vector comprising the same, a composition for regulating gene expression by electromagnetic fields comprising the vector, or a method for regulating gene expression. The present invention also relates to a vector comprising an Lgr4 gene promoter regulated by electromagnetic fields or a fragment thereof, and a cellular reprogramming gene; a composition for cellular reprogramming comprising the vector; a method for cellular reprogramming; and a method for cell therapy or gene therapy using the vector.

### TECHNICAL FIELD

The present invention relates to a promoter of Lgr4 gene regulated by electromagnetic fields or a fragment thereof, a vector comprising the same, a composition for regulating gene expression by electromagnetic fields comprising the vector, or a method for regulating gene expression. The present invention also relates to a vector comprising a promoter of the Lgr4 gene or a fragment thereof, regulated by electromagnetic fields, in combination with a cellular reprogramming gene; a composition for cellular reprogramming comprising the vector; a method for cellular reprogramming; and a method for cell therapy or gene therapy using the vector.

### BACKGROUND ART

One of the most commonly used methods for gene overexpression is a method which utilizes the sequence of an intron region of a gene called the promoter to express a target gene. A gene “promoter” refers to a DNA region that includes binding sites for RNA polymerase, enhancers, or other elements necessary for the expression of a downstream target gene. The promoter is located near the region responsible for the transcription of the target gene.

Methods utilizing such promoter sequences to overexpress target genes are typically employed. Promoters with superior gene expression-inducing abilities, such as the CMV promoter, EF1α promoter, PGK promoter, and U6 promoter, have been commonly utilized. Furthermore, techniques enhancing the gene expression ability of a given promoter have been proposed, in which a mutation is introduced into the promoter sequence or by linking the target gene with a promoter with excellent gene expression-inducing ability.

However, these methods for inducing gene expression utilizing promoters have limitations in their various applications, particularly due to difficulties in regulating in vivo permeability and the on-and-off control of gene expression when inducing gene expression in vivo. For instance, the difficulties in regulating in vivo gene expression in specific organs significantly limit applications in cell regeneration and cell and gene therapies.

In order to solve these issues, methods for regulating gene expression using promoters activated by chemicals, such as doxycycline, have been developed. However, these methods also have limitations in use due to the challenges in in vivo delivery of such chemicals and the lack of response to these chemicals under various biological conditions. Additionally, with advances in optogenetics, which utilizes light, techniques for light-responsive gene expression have been developed. However, due to the difficulties in the regulation of in vivo light penetration, these techniques are also limited in their application in in vivo cell regeneration or cell and gene therapies.

Accordingly, the present inventors have made many efforts to discover promoters that can be regulated by electromagnetic field exposure from the aspect of magnetogenetics, and have continuously conducted research to discover techniques for regulating gene expression using these promoters. As a result, the present inventors have already obtained a patent from a previously-filed application, which relates to a technique for regulating target gene expression using the Egr1 gene promoter or the Ifi44 promoter, both of which are electromagnetic field-responsive promoters (see KR 10-2128032 B1).

### DISCLOSURE

### Technical Problem

However, for the utilization and application of such electromagnetic field-responsive gene promoters in cell and gene therapies, it is essential to develop sensitive electromagnetic field-responsive gene promoters that exhibit relatively low basal levels and respond with higher efficiency under electromagnetic field (EMF) conditions. Additionally, there still remains a need to develop new promoters that are sensitive to the on-and-off of gene expression, function effectively both in vitro and in vivo, and are applicable to a variety of target genes.

### Technical Solution

Under the circumstances, the present inventors have newly discovered the Lgr4 gene promoter and fragments thereof that are regulated by electromagnetic fields, and the present inventors have confirmed that these promoters are capable of exhibiting significantly superior and more efficient responsiveness to the on-and-off control of the electromagnetic fields, compared to the previously developed Egr1 gene promoter or Ifi44 promoter, thereby providing the present invention.

An embodiment of the present invention provides an isolated nucleic acid corresponding to a fragment of an Lgr4 gene promoter sequence that exhibits superior promoter activity regulated by electromagnetic fields.

Another embodiment of the present invention provides a vector comprising the Lgr4 gene promoter or a fragment thereof. In one embodiment, the Lgr4 gene promoter is represented by the nucleotide sequence of SEQ ID NO: 1 or SEQ ID NO: 2. The fragment thereof is a fragment comprising the nucleotides from position 1337 to position 1978 of the sequence of SEQ ID NO: 1 or a fragment of SEQ ID NO: 2 corresponding thereto. The Lgr4 gene promoter or a fragment thereof exhibits promoter activity regulated by electromagnetic fields.

TABLE 1

| | |
|---|---|
| Mouse Lgr4 | aggagttgtg ttttttaaga aagaagattt tagctgggca tggtggcaca cgcctttaat 60 |
| gene | cctagcactt gagaggcaga ggcaggcaaa tttctgagtt cgaggccagc atggtctaga 120 |
| promoter | aagtgagtgc caggacagcc agggctatac agagaaaccc tgtctcaaaa aaccaaaaaa 180 |
| sequence | aaaaaaaaaa aaaaaaaaaa aaagacttta agtatcaatt agaattagac tttttcctca 240 |
| (SEQ ID | ctttatttac tcatgaaatt ggctgaaaag taatagaaat ggaaaaacaa aaacaaaaaa 300 |
| NO: 1) | caagttgcgg aggcttctga aatactaact agaccaggag tgagatgatc ttgggcagag 360 |
| | ggcagaagaa catggtatcc aagagattct gaggccttgt ctataaagtc tgagcgccac 420 |
| | tggtctttct ttttaaagtt ctctttcaac cttatagtga ggaaattcat ctttottaaa 480 |
| | acttgtctgt atttatttat ccattagcat aactttagag tatatggatt caacctgact 540 |
| | ttattattat ttagcatatt ctttaatgta gagatggtag attttttttt ttttatttag 600 |
| | gaaatcggct agcttttcgt taatttattt acattttctt ttttttttat ttgcatgagg 660 |
| | tggctagtta actttataca gtatagatat tttaatgctc ttcttgctct aatgtaatgg 720 |
| | gttcattaaa aatgtgtaca atctagagta aaaacaacca aaggcctagg agaaagcaag 780 |
| | aaaagaggca ctgagaatgc tgattttttt ttttctataa acattatgtg ggaaaaagga 840 |
| | agggatgctg atagttaaca cacaagttgt ttctggtgtc tcaggggcag tcagctggca 900 |
| | ctcaaatgtt ctcttccagt gctggttaac cggcacctcc ttggctggac tcctcaacat 960 |
| | ccttgtgttt agggagaaag aagtctgatt ggtgtcatgg atgattaacc gctaatttag 1020 |
| | ctcccaagtg tgaatgaaaa agggtactaa gatctcagag gtgatgtgag atacaggagg 1080 |
| | ggtcacgctg ttttaattta gtgcacggtt gaactcaaaa atttggatca gatagaaaga 1140 |
| | aatgtttctc cctccttttc gacccccacc cccaccccca tttctttcct ctgtttgcaa 1200 |
| | agctctcagt gcccttgccc ttaagaacag cgaagagtca tttgacacgt ctgaaagctg 1260 |
| | gaggcgagtt ttacttttca ggtcatccaa tttaggagat ccaatttctg cggaggggag 1320 |
| | aaaaaaactg ggtgggggag ggaaacagca gactcctggt cttccgatct gtctaccttc 1380 |
| | aatacacaac ctgacatgca gatccagcca agtcagggct tttacttgaa cctccactac 1440 |
| | cagccccagc agcagcagca caaccttgtc actcattcca gagaaacacg ccccattcct 1500 |
| | cttgaccaat aatggctcca ctgcctgcat agtaatgagc tcgagacctc ccctgaccaa 1560 |
| | tagcgctccc ggagcggggt tagttttgca tgtacctaaa tgatttgcat aacccggcgg 1620 |
| | ccaggggctc ccgaggcgag cgtgcaaccc tagaagggaa aaggacgcgc ggagcgggag 1680 |
| | ccgcctcggg gagagcgcgg acaaccaggg tgtttgtgag agctggggcg ggggttggga 1740 |
| | cgcctggccg gcatggctgg ccgcaacctt aggctgcgct gaggagctgt gcggctggag 1800 |
| | gaggcccggg acaggaggcg gcggcgatgg cagcgcgcgg cccgggcagc cgctctgggc 1860 |
| | cgggtcggct ggcctgagcc gcggggctgc cggtgcgcgt ccatggagca gcgggaaggg 1920 |
| | agaaactgcg gagcgccgcg tcctaacgct ccggcggcag actgctgaag gaagcgag |
| Human Lgr4 | gtagaaccga agtccagctt atccatcagt caacaagtcc tgtgtgctct gcacaaagac 60 |
| gene | accaaggcaa gtatcactac gatagggtac tttcttcatg gcccataagc tccatgccac 120 |
| promoter | atatgcagag gggttgcagc ccagttctat tttttatcta gcctgtggat gccgcaaatt 180 |
| sequence | tacttacaca caaccaatga aaactttaag aggaaccgat ctgtagacag agaccctatt 240 |
| (SEQ ID | attttttgct tgccgtcaat ttttatttgg tttatctcag tatctttcca gtccttatga 300 |
| NO: 2) | tttatttaag agctgatcat ttttctctag ttatctgaga tattacctta atcactggtt 360 |
| | ttttgttttt tgtctttata ctttcttggg acatcttatc caagagatgg agactaattg 420 |
| | accaatgtat ctctaatgca aagcatacaa aatgtaccaa ataagaaata caaacacttc 480 |
| | aatatatctt atcttttctg ttccctaccc aagtcttttg ttcctgatct tttatcttct 540 |
| | tggctaagct gcaaatttga atcttccttt cttacatttg tcatctaata gacaatagga 600 |
| | atttattttt tagtaaaatc tgcatctccc cttttttcaa aatataaatc tctcctttca 660 |
| | cctaagtttt cagccataag attttattat ttgaagacaa agaataaagt tctccaagtg 720 |
| | tggttcatga atcacctgtg tcactttcac ttgctgtgca acaaaatgct ggtttctggg 780 |
| | ccccacttag gcctactaaa tcattctgca tttttaatca agatttgcat ataagccgga 840 |
| | ctccgtggct catgactgta atcccagcac tttgggaggc caaggcaggt ggatcacttg 900 |
| | aggtgaggag tttgaggcca gcctggccaa catgagaaac cccatctcta ctaaaaaaaa 960 |
| | aaaaaaaaaa aaaaaaaaaa aagtagctaa acatggtggt gggcacctgt aataccagct 1020 |
| | actggggagg ctgaggcagg agaatcactt gaacccggga ggtggaggtt gcagtgagcc 1080 |
| | aagatcacgc cactggacac agtgagtgag agtccgtttc aaaaaaaaaa aaaaaaaaga 1140 |
| | agagatctta aagaaacgat tacagaaact aactttacaa tttcttcctt atttgttcat 1200 |
| | gacagttgct agaaaataat ggaaatgatt tagacgatga aagcctattt agggaaggct 1260 |
| | ctaagagtac cagattagga gccaggagcc caggagctgt tccgtgatga tgttgggcag 1320 |
| | aacacactct ctctcgagcc tcagatttct tatctataaa accagagttt tctgggggtc 1380 |
| | tttaagttgt ctttcagccc tgcagtgaga ggagatatgt cattcctaaa acttgaaatg 1440 |
| | aaggcatatg tttatttatt aactctctag cttctagaca gatttactgt atgtgactta 1500 |
| | tgtgcccaac ctgacattat cttttagata aactcccaaa tacaaagatg ataaaatttt 1560 |
| | ttcattttaa acttataggt ctgcatcaac ctgtttccat tgtcctaagt tttatttaca 1620 |
| | tacgatggct agtcaacttt atatgataat tatctttaaa tcctgctttt tttttttttt 1680 |
| | tttttttttt tttttgagac agagttttgc tcttgttgcc caggctggaa tgcaatggcg 1740 |
| | cgatctccag ctcaccgcaa cctccgcctc ccgggctcaa gcaattcgcc tgcctcagcc 1800 |
| | tcccgagtag ctgggattac aggcatctgc caccacaccc ggctaatttt gtatttttag 1860 |
| | tagagacggg gtttcttcat gttggtcagg gtggtctaga actcccaacc tcaggtgatc 1920 |
| | cgcccgccgc agcctcccaa agtgttggga ttacaggcgt gagccaccgc gcccggccga 1980 |

Another embodiment of the present invention provides a composition for regulating gene expression by electromagnetic fields comprising the vector.

Another embodiment of the present invention provides a method for regulating gene expression, comprising: introducing the vector into a cell; and applying electromagnetic fields to the cell or blocking the electromagnetic fields from acting on the cell.

Another embodiment of the present invention provides a vector comprising a promoter of the Lgr4 gene or a fragment thereof, and a cellular reprogramming gene operably linked thereto. In one embodiment, the Lgr4 gene promoter is represented by the nucleotide sequence of SEQ ID NO: 1 or SEQ ID NO: 2. The fragment thereof is a fragment comprising the nucleotides from position 1337 to position 1978 of the sequence of SEQ ID NO: 1 or a corresponding fragment of SEQ ID NO: 2. The Lgr4 gene promoter or a fragment thereof exhibits promoter activity regulated by electromagnetic fields.

Another embodiment of the present invention provides a composition for cellular reprogramming by electromagnetic fields comprising the vector.

Another embodiment of the present invention provides a method for cellular reprogramming comprising: introducing the vector into a cell; and applying electromagnetic fields to the cell.

### BRIEF DESCRIPTION OF THE DRAWINGS

FIG. 1 is a diagram illustrating the qRT-PCR measurement results showing the expression of the Lgr4 gene induced by EMF exposure in various tissues.

FIG. 2 is a schematic diagram illustrating the structure of a vector comprising the Lgr4 promoter, and the luciferase gene or GFP gene, and its activation by EMF exposure.

FIG. 3 is a diagram illustrating the results of luminescence assays comparing cell-specific gene induction responsiveness in various cell groups following the introduction of a vector comprising the Lgr4 promoter and luciferase gene.

FIG. 4 is a diagram illustrating the Western blotting and FACS results comparing GFP gene expression induced by EMF exposure following the introduction of a vector comprising the Lgr4 promoter and GFP gene into cells.

FIG. 5 is a diagram illustrating the Western blotting and FACS results comparing GFP gene expression following the removal of EMF exposure following the introduction of a vector comprising the Lgr4 promoter and GFP gene into cells.

FIG. 6 is a diagram illustrating the measurements of in vivo GFP expression in a transgenic mouse model constructed using a vector comprising the Lgr4 promoter and GFP gene, following EMF exposure.

FIG. 7 is a diagram illustrating the FACS results comparing the GFP expression levels obtained by the introduction of a vector comprising the Lgr4 promoter and GFP gene into cells with the GFP expression levels obtained from the vectors comprising the Egr1 or Ifi44 promoter and GFP gene.

FIG. 8 is a schematic diagram illustrating vectors each comprising the Lgr4 promoter and Oct4, Sox2, c-Myc, or Klf4 gene, and their respective activity.

FIG. 9 is a diagram illustrating the Western blotting results confirming the expression of Oct4 gene following the injection of vectors comprising the Lgr4 promoter and Oct4, Nanog, Sox2, c-Myc, and Klf4 (OSKM) genes into the tail vein of mice, with and without EMF exposure.

FIG. 10 is a diagram illustrating the qRT-PCR and Western blotting measurement results showing the expression levels of the pluripotency genes Oct4, Nanog, Sox2, c-Myc, and Klf4 with and without EMF exposure.

FIG. 11 is a diagram illustrating the results confirming the pluripotency of induced pluripotent stem cells (iPSCs) generated by applying EMF to cells transfected with vectors comprising the Lgr4 promoter and the Oct4, Nanog, Sox2, c-Myc, and Klf4 genes through the number of AP-positive cells and immunostaining for Nanog and Oct4.

FIG. 12 is a schematic diagram illustrating vectors comprising the Lgr4 promoter and Ascl1, Pitx3, Nurr1, or Lmx1a (APNL) genes and confirming the expression of each gene following EMF treatment.

FIG. 13 is a diagram illustrating the results of qRT-PCR and immunostaining measuring the expression levels of TH, Dat, Pitx3, NeuroD1, Tuj1, and Map2 genes, all of which are dopaminergic neuron marker genes, with or without EMF exposure.

FIG. 14 is a diagram illustrating the results of a test assessing the survival ability of aged mouse models injected with vectors comprising the Lgr4 promoter and the Oct4, Sox2, c-Myc, and Klf4 (OSKM) genes, followed by transient EMF exposure to induce the expression of pluripotency genes.

FIG. 15 is a diagram illustrating the results of a study in which vectors comprising the Lgr4 promoter and the Ascl1, Pitx3, Nurr1, or Lmx1a (APNL) genes were injected into MPTP-induced Parkinson's disease mouse models, confirming the presence of directly reprogrammed induced dopaminergic neurons and examining the recovered behavioral patterns of the mice.

FIG. 16 is a diagram illustrating luminescence assay results comparing the reporter gene induction responsiveness to EMF exposure by the introduction of a vector comprising the human Lgr4 promoter and luciferase gene into human fibroblasts.

### DETAILED DESCRIPTION OF PREFERRED EMBODIMENTS

An aspect of the present invention provides an isolated nucleic acid having promoter activity regulated by electromagnetic fields, comprising the nucleotides from position 1337 to position 1978 of a promoter sequence of Lgr4 gene of SEQ ID NO: 1 or a fragment of SEQ ID NO: 2 corresponding thereto.

Another aspect of the present invention provides a vector comprising a nucleic acid having promoter activity regulated by electromagnetic fields, comprising a promoter sequence of Lgr4 gene or a fragment thereof. In a specific embodiment, the promoter of the Lgr4 gene is represented by the nucleotide sequence of SEQ ID NO: 1 or SEQ ID NO: 2. The fragment thereof is a fragment comprising the nucleotides from position 1337 to position 1978 of SEQ ID NO: 1 or a fragment of SEQ ID NO: 2 corresponding thereto.

In one embodiment, the vector may further comprise a target gene operably linked to the promoter nucleic acid.

Another aspect of the present invention provides a composition for regulating gene expression by electromagnetic fields comprising the vector.

Another aspect of the present invention provides a method for regulating gene expression, comprising: introducing the vector into a cell; and applying electromagnetic fields to the cell or blocking the electromagnetic fields from acting on the cell.

In one embodiment, the application of electromagnetic fields to the composition or in the method may induce the expression of a gene, while the blockage of the electromagnetic fields may reduce the expression of the gene.

In one embodiment, the composition or method may be used in gene therapy or cell therapy.

In one embodiment, the electromagnetic fields may be applied with an intensity of 10 G or more and 30 G or less, or at a frequency of 50 Hz or more and 300 Hz or less.

In one embodiment, the gene with regulated expression is a cell reprogramming gene, and the application of electromagnetic fields may express the gene to induce cellular reprogramming.

In one embodiment, the cellular reprogramming gene is one or more selected from the group consisting of Oct4, Nanog, Sox2, c-Myc, Klf4, Lin28, and L-Myc, and the application of electromagnetic fields may express the gene to induce reprogramming of somatic cells into induced pluripotent stem cells.

In one embodiment, the cellular reprogramming gene is one or more selected from the group consisting of Ascl1, Nurr1, Pitx3, and Lmx1a, and the application of electromagnetic fields may induce the expression of the gene to promote reprogramming of somatic cells into neurons.

Another aspect of the present invention provides a vector comprising a promoter of the Lgr4 gene or a fragment thereof, and a cellular reprogramming gene operably linked thereto, in which the Lgr4 gene promoter is represented by the nucleotide sequence of SEQ ID NO: 1 or SEQ ID NO: 2. A fragment thereof is a fragment comprising the nucleotides from position 1337 to position 1978 of the sequence of SEQ ID NO: 1 or a corresponding fragment of SEQ ID NO: 2, and the Lgr4 gene promoter or a fragment thereof exhibits promoter activity regulated by electromagnetic fields.

Another aspect of the present invention provides a composition for cellular reprogramming by electromagnetic fields comprising the vector.

Another aspect of the present invention provides a method for cellular reprogramming, comprising: introducing the vector into a cell; and applying electromagnetic fields to the cell.

Hereinafter, the present invention is described in more detail.

As used in this specification and the claims, the terms “comprise”, “comprises”, “comprised”, and “comprising” are interpreted as specifying the presence of the stated features, integers, steps, or components; however, it will be interpreted that these terms do not exclude the presence of one or more other features, integers, steps, components, or combinations thereof.

In the present specification, descriptions related to documents, statutes, materials, devices, articles, and the like are included solely for the purpose of providing context to the present invention. All or part of the same does not form part of the basis of conventional technology, not does it suggest or represent common knowledge in the art prior to the priority date of each claim of the present invention.

The present invention relates to a technique for regulating gene expression using an electromagnetic field-responsive promoter. In the present invention, the electromagnetic field-responsive promoter functions as a gene expression “switch” when introduced into a cell along with a target gene operably linked to the promoter, thereby inducing gene expression upon the application of electromagnetic fields and ceasing the induction of gene expression upon the blockage of the electromagnetic fields.

Accordingly, the present invention provides a non-invasive method for regulating gene expression during gene therapy or cell therapy. The present invention can provide a safer, more effective, and more precise method for gene therapy or cell therapy by inducing the production of a desired gene product for a desired period of time. For example, the present invention may be used in disease treatment by providing a patient with genetic deficiencies with the gene product the patient lacks. Alternatively, the present invention may be used in disease treatment by introducing a reprogramming factor into a cell to reprogram the cell into a specific type of cell.

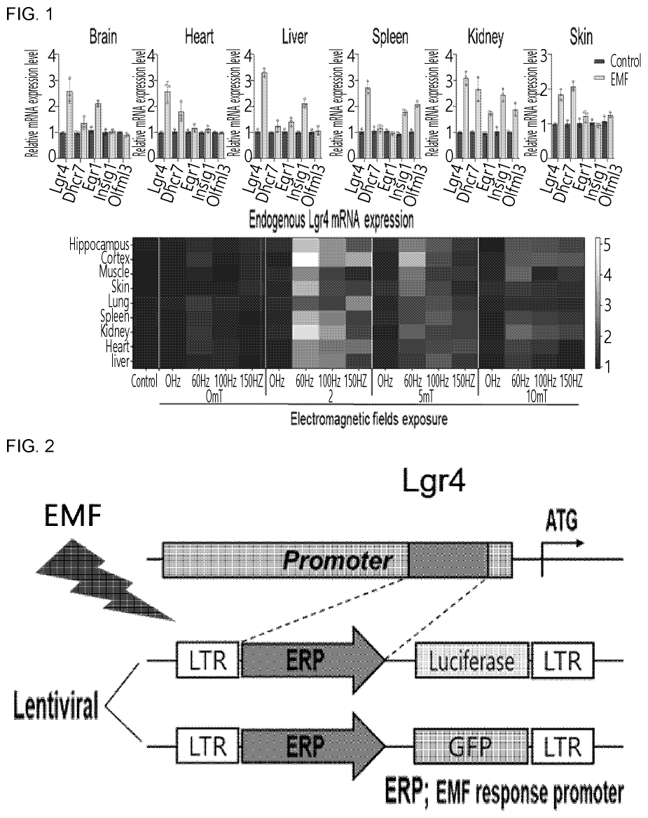

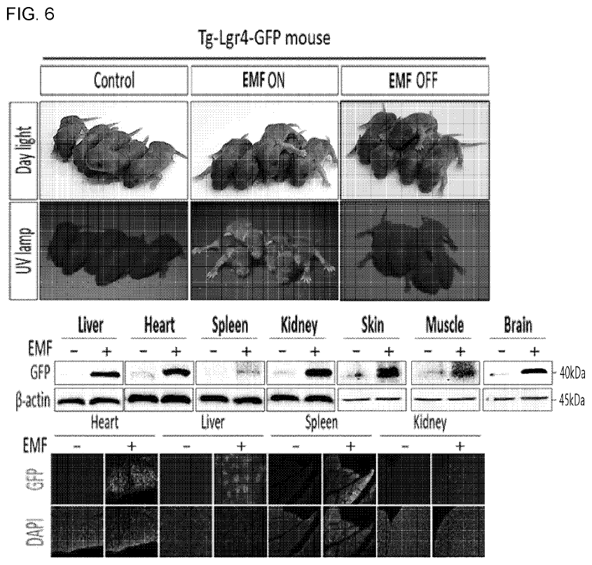

Specifically, the present invention has selected Lgr4 gene as the gene in which the expression level changes in tissues of various organs due to electromagnetic field exposure. In response to the electromagnetic field exposure, the Lgr4 gene showed increase in gene expression level in all tested tissues, including the brain, hippocampus, cerebral cortex, heart, liver, spleen, kidney, skin, muscle, and lungs, and exhibited significantly superior responsiveness compared to the control group without the application of electromagnetic fields (FIG. 1). Accordingly, a vector was constructed by operably linking a reporter gene (e.g., a luciferase gene or GFP gene) downstream of a promoter of the Lgr4 gene to verify whether a promoter of the Lgr4 gene responds to electromagnetic field exposure (FIG. 2). This vector was introduced into various somatic cells, and gene expression in response to the electromagnetic field exposure was measured using luminescence or fluorescence. As a result, the Lgr4 promoter was activated by the electromagnetic field exposure, thereby inducing gene expression, and gene expression ceased upon the blockage of the electromagnetic fields, confirming that the gene expression is precisely regulated by the on-and-off of electromagnetic fields (FIGS. 3 to 5). Furthermore, it was confirmed that the gene expression can also be regulated by the on-and-off of the electromagnetic fields in in vivo experiments using animal models (FIG. 6). Specifically, the Lgr4 gene promoter of the present invention was significantly superior compared to Egr1 gene promoter and Ifi44 promoter, which are electromagnetic field-responsive promoters previously provided by the present inventors, in gene expression regulation in response to the on-and-off of the electromagnetic fields.

Accordingly, one embodiment of the present invention provides an isolated nucleic acid having promoter activity regulated by electromagnetic fields, comprising all or part of the sequence of a promoter of the Lgr4 gene. Specifically, the present invention provides an isolated nucleic acid having promoter activity regulated by electromagnetic fields, comprising the nucleotides from position 1337 to position 1978 of the promoter sequence of the Lgr4 gene of SEQ ID NO: 1. Further, the present invention provides an isolated nucleic acid having promoter activity regulated by electromagnetic fields, comprising a portion of the nucleotides of SEQ ID NO: 2 that corresponds to the nucleotides from position 1337 to position 1978 of the promoter sequence of the Lgr4 gene of SEQ ID NO: 1.

As used herein, the term “isolated” means that the substance is separated from the natural environment in which the substance occurred. For example, a nucleic acid or peptide that naturally exists in an organism is not “isolated”, whereas a nucleic acid or peptide separated from naturally-coexisting substances is considered “isolated”. Further, a nucleic acid or peptide introduced into an organism by transformation, gene editing, or other recombination methods is considered “isolated”, even if it exists within the organism.

As used herein, the terms “nucleic acid”, “nucleic acid molecule”, and “nucleic acid sequence” refer to a DNA or RNA molecule or sequence. The term “isolated nucleic acid” includes, for example, isolated DNA, isolated PCR products, isolated mRNA, cDNA, or restriction enzyme fragments. Further, an isolated nucleic acid molecule includes, for example, sequences inserted into vectors (viral vectors, episomal vectors, plasmid vectors, cosmid vectors, etc.) or into artificial chromosomes. An isolated nucleic acid is preferably cleaved from a genome in which the nucleic acid is found. Further, an isolated nucleic acid is preferably no longer linked to a non-regulatory sequence, a non-coding sequence, or another gene located upstream or downstream of the nucleic acid molecule when found within the genome.

As used herein, the term “promoter” refers to an untranslated nucleic acid, which comprises a site to which RNA polymerase binds and has activity of initiating transcription of a structural gene located downstream of the binding site. In eukaryotes, the promoter further comprises binding sites for proteins called transcription factors, which regulate the binding of RNA polymerase and participate in the transcription process. On the other hand, in prokaryotes, the promoter is commonly defined as the binding site for RNA polymerase located near the transcription start site.

For the object of the present invention, a promoter refers to a promoter having activity regulated by electromagnetic fields. For example, the promoter is a nucleic acid fragment comprising all or part of the sequence of an Lgr4 gene promoter. As used herein, the term “fragment” refers to a region that is shorter than the reference nucleic acid sequence but retains a substantially identical biological function or activity to that of the reference nucleic acid.

For example, the full sequence of an Lgr4 gene promoter comprises the nucleic acid sequence represented by SEQ ID NO: 1 (mouse) or SEQ ID NO: 2 (human). Further, a fragment of an Lgr4 gene promoter comprises part of the full sequence of the Lgr4 gene promoter which have promoter activity regulated by electromagnetic fields even when isolated. For example, the fragment of an Lgr4 gene promoter may be an isolated nucleic acid molecule comprising the nucleotides from position 1337 to position 1978 of the promoter sequence of the Lgr4 gene of SEQ ID NO: 1 (mouse), or an isolated nucleic acid molecule comprising the nucleotides of the promoter sequence of the Lgr4 gene of SEQ ID NO: 2 (human) corresponding to the nucleotides of SEQ ID NO: 1. In the present invention, the sequence consisting of the nucleotides from position 1337 to position 1978 of the promoter sequence of the Lgr4 gene of SEQ ID NO: 1 is described in SEQ ID NO: 3 below.

TABLE 2

| | |
|---|---|
| SEQ ID | GGAGGGAAACAGCAGACTCCTGGTCTTCCG |
| NO: 3 | ATCTGTCTACCTTCAATACACAACCTGACA |
| | TGCAGATCCAGCCAAGTCAGGGCTTTTACT |
| | TGAACCTCCACTACCAGCCCCAGCAGCAGC |
| | AGCACAACCTTGTCACTCATTCCAGAGAAA |
| | CACGCCCCATTCCTCTTGACCAATAATGGC |
| | TCCACTGCCTGCATAGTAATGAGCTCGAGA |
| | CCTCCCCTGACCAATAGCGCTCCCGGAGCG |
| | GGGTTAGTTTTGCATGTACCTAAATGATTT |
| | GCATAACCCGGCGGCCAGGGGCTCCCGAGG |
| | CGAGCGTGCAACCCTAGAAGGGAAAAGGAC |
| | GCGCGGAGCGGGAGCCGCCTCGGGGAGAGC |
| | GCGGACAACCAGGGTGTTTGTGAGAGCTGG |
| | GGGGGGGGTTGGGACGCCTGGCCGGCATGG |
| | CTGGAGGCTGCGCTCCGCAACCTTGAGGAG |
| | CTGTGCGGCTGGAGGAGGCCCGGGACAGGA |
| | GGCGGCGGCGATGGCAGCGCGCGGCCCGGG |
| | CAGCCGCTCTGGGCCGGGTCGGCTGGCCTG |
| | AGCCGCGGGGCTGCCGGTGCGCGTCCATGG |
| | AGCAGCGGGAAGGGAGAAACTGCGGAGCGC |
| | CGCGTCCTAACGCTCCGGCGGCAGACTGCT |
| | GAAGGAAGCGAG |

The present invention further includes a nucleic acid having at least 80%, for example, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the nucleic acid sequence of SEQ ID NO: 1, SEQ ID NO: 2, or SEQ ID NO: 3, and having a promoter activity substantially identical to the nucleic acid of SEQ ID NO: 1, SEQ ID NO: 2, or SEQ ID NO: 3.

The term “% identity” of a sequence refers to the degree to which the nucleotides are identical when two or more nucleic acid sequences are aligned for maximum correspondence and compared. The percent sequence identity can be calculated, for example, by comparing two sequences aligned for maximum correspondence of the entire comparison region, determining the number of position at which identical amino acids or nucleic acids occur in both sequences and thereby obtaining the number of matched positions, dividing the number of matched positions by the total number of positions in the comparison region (i.e., range size), and multiplying the number by 100 to obtain the percentage of sequence identity. The percent sequence identity may be determined using a known sequence comparison program, such as the BLAST program family (Altschul S F et al., J Mol Biol, 215, 403-410, 1990; Altschul S F et al., Nucleic Acids Res., 25:3389-3402, 1997; available from the National Center for Biotechnology Information (NCBI) and accessible via the NCBI website at www.ncbi.nlm.nih.gov), FASTA (Pearson W R, Methods in Enzymology, 183, 63-99, 1990; Pearson W R and Lipman D J, Proc Nat Acad Sci USA, 85, 2444-2448, 1998; available as part of the Wisconsin Sequence Analysis Package), or Wisconsin Sequence Analysis Package version 9.1 (Wisconsin Sequence Analysis package, version 9.1; Devereux J et al., Nucleic Acids Res, 12, 387-395, 1984, accessible from the Genetics Computer Group in Madison, Wisconsin, USA), including BESTFIT and GAP.

As used herein, the term “electromagnetic field (EMF)” refers to a wave composed of electric and magnetic fields. It is characterized in that when an electric field changes with time in space, a magnetic field is generated in the surrounding environment, and when a magnetic field changes with time in space, an electric field is generated in the surrounding environment, propagating through space at the speed of light. An electric field refers to the space in which the electric force acts perpendicularly, typically expressed in volts per meter (V/m), while a magnetic field refers to the space in which magnetic force acts horizontally, typically measured in Gauss (G) or Tesla (T). Electromagnetic fields can be categorized in order of increasing wavelength as gamma rays, X-rays, ultraviolet rays, visible light, infrared rays, and radio waves (including longwave, medium wave, shortwave, ultrashort wave, extremely high frequency, and microwaves). Based on the frequency (i.e., the number of oscillations per second), electromagnetic fields can be categorized into Extremely Low Frequency (ELF; 0 KHz to 1 kHz), Very Low Frequency (VLF; 1 kHz to 500 kHz), Radio Frequency (RF; 500 KHz to 300 MHz), Microwaves (MW; 400 MHz to 300GHz), etc.

The frequency of the electromagnetic fields used herein may be from 30 Hz to 500 Hz, and specifically from 50 Hz to 300 Hz, but is not limited thereto. The strength of the electromagnetic fields may be from 5 G to 50 G, and specifically from 10 G to 30 G, but is not limited thereto.

As provided herein, an Lgr4 promoter, which is an electromagnetic field-responsive promoter, or a fragment thereof is characterized by its activation upon the application of electromagnetic fields, which induces the expression of a gene operably linked downstream, and inactivation upon the blockage of electromagnetic fields, which reduces or ceases gene expression.

The promoter of the present invention may be cloned into a vector and readily transferred into a cell or organism.

Accordingly, an embodiment of the present invention provides a vector comprising a nucleic acid having promoter activity regulated by electromagnetic fields, comprising an Lgr4 gene promoter or a fragment thereof.

As used herein, the term “vector” refers to a gene construct comprising essential regulatory elements operably linked to a gene insert encoding a target protein such that the gene insert is expressed within a cell of a subject.

As used herein, the term “operably linked” means that the linkage between nucleic acid sequences is functionally associated. For example, a coding sequence (e.g., a sequence encoding a target protein) may be operably linked to suitable regulatory elements to enable replication, transcription, and/or translation of the coding sequence. For example, the coding sequence is operably linked to a promoter when the promoter is capable of promoting the transcription of the coding sequence. The regulatory elements do not need to be close to the coding sequence as long as they function properly. For example, an intervening sequence that is transcribed but not translated may exist between the promoter sequence and the coding sequence, and the promoter sequence may still be considered operably linked to the coding sequence. An operable linkage may be achieved using gene recombination techniques known in the art, and site-specific DNA cleavage and ligation may be performed using enzymes such as restriction enzymes and ligases known in the art. For example, the operable linkage between each component in a vector may be achieved by ligation at convenient restriction enzyme sites. If no such sites are present, it may be achieved using synthetic oligonucleotide adaptors or linkers according to conventional methods, but the method of achieving an operable linkage is not limited thereto.

Vectors, when introduced into cells, may either irreversibly integrate into the genome of the host cell or remain non-integrated and respond to electromagnetic fields to regulate gene expression. Such vectors may include transcriptional and translational expression regulatory sequences that enable the expression of the gene in a selected host. The expression regulatory sequences may include any operator sequences for regulating transcription and/or sequences for controlling the termination of transcription and translation. Start codons and stop codons are typically considered part of the nucleic acid sequence encoding the target protein and must function properly in the organism and be in-frame with the coding sequence when the vector is introduced. Further, if the vector is a replicable expression vector, the vector may include an origin of replication. Furthermore, it may appropriately include enhancers, untranslated regions at the 3′ end of the target gene, selection markers (e.g., antibiotic resistance markers), replication units, or the like. Vectors may either self-replicate or integrate into the host genomic DNA. For example, the vector includes various forms, such as plasmids, viral vectors, episomal vectors, bacteriophage vectors, or cosmid vectors.

The vector of the present invention may be introduced into cells using a viral vector for the purpose of ultimate use in gene therapy or cell therapy. Examples of viral vectors include vectors derived from lentiviruses, retroviruses (e.g., human immunodeficiency virus (HIV), murine leukemia virus (MLV), Rous sarcoma virus (RSV), or mouse mammary tumor virus (MMTV)), adenoviruses, adeno-associated viruses, and herpes simplex viruses, but are not limited thereto.

In a specific embodiment, the term “lentiviral vector” refers to a viral vector or plasmid that primarily contains structural and functional genetic elements including LTRs derived from lentiviruses. For example, the lentiviral vector may refer to a vector derived from at least part of a lentiviral genome, comprising self-inactivating lentiviral vectors, such as those provided in Milone et al., Mol. Ther. 17(8): 1453-1464 (2009). Other examples of the lentiviral vectors that may be used in clinical applications include the LENTIVECTOR™ gene delivery technology from Oxford BioMedica and the LENTIMAX™ vector system from Lentigen, but are not limited thereto.

An example of non-viral vectors is episomal vectors, which are non-viral, non-integrating vectors known to have the feature of expressing genes included in the vector without being inserted into the chromosome. The cell including the episomal vector includes the cases where the episomal vector is inserted into the genome and where the episomal vector exists within the cell without being inserted into the genome.

The vector of the present invention may further include a target gene operably linked to the promoter of the present invention.

As used herein, the term “target gene” is used interchangeably with “desired gene” and the like, and the term refers to a gene which is operably linked to the downstream of a promoter, which its expression regulated by the promoter. The target gene may refer to a nucleic acid sequence or exogenous nucleic acid sequence of a specific length that encodes the desired product to be expressed, such as RNA or protein. The target gene is not particularly limited and includes genes encoding exogenous proteins, endogenous proteins, or reporter proteins. The target gene may encode proteins in their natural or mutant form. Exogenous proteins refer to proteins that do not naturally exist in a specific tissue or cell, whereas endogenous proteins refer to proteins expressed by genes that naturally exist in a specific tissue or cell. Further, reporter proteins refer to marker proteins expressed by reporter genes and used to quantify or detect expression or activity of reporter genes within a cell based on the presence of reporter genes. The sequence of a target gene may be in a truncated form, a fused form, or a tagged form, and may be cDNA or gDNA, but is not limited thereto.

The target gene may be one, or two or more. When there are two or more target genes, vectors may be used either by constructing separate vectors for each gene, with one target gene connected to one electromagnetic field-responsive promoter, or by constructing vectors with two or more target genes connected to one electromagnetic field-responsive promoter in a polycistronic form.

As used herein, the term “polycistronic”, or the compatible term “bicistronic”, refers to a system in which ribosomes can synthesize polypeptides from within the mRNA in eukaryotic cells, enabling the synthesis of multiple polypeptides from a single mRNA. Typically, prokaryotic cells have a polycistronic system in which ribosomes can bind to different positions of one mRNA to synthesize multiple proteins at once. However, eukaryotic cells fundamentally have a monocistronic system in which a single promoter produces one mRNA that is translated to synthesize a polypeptide. In one embodiment of the present invention, vectors were constructed such that the cellular reprogramming genes Oct4, Nanog, Sox2, c-Myc, and Klf4 are linked in a polycistronic form downstream of the Lgr4 promoter, thereby enabling simultaneous expression from a single transcript.

In the present invention, the electromagnetic field-responsive promoter functions as a gene expression “switch” when introduced into a cell along with a target gene operably linked to the promoter, inducing gene expression upon the application of electromagnetic fields and ceasing the induction of gene expression upon blockage of electromagnetic fields. Accordingly, vectors comprising electromagnetic field-responsive promoters may be used to regulate gene expression by electromagnetic field.

In another embodiment of the present invention, the present invention provides use of said vector in regulating gene expression by electromagnetic fields. Specifically, the present invention provides a composition for regulating gene expression by electromagnetic fields comprising the vector. Further, the present invention provides a method for regulating gene expression, comprising: introducing said vector into a cell; and applying or blocking electromagnetic fields to the cell.

These compositions and methods regulate gene expression in a manner where the gene is expressed upon the application of electromagnetic fields and reduced or ceased upon the blockage of electromagnetic fields.

Additionally, the present invention can be applied to various gene therapy or cell therapy techniques, depending on the target gene to be regulated. As used herein, the term “gene therapy” refers to the treatment of diseases by utilizing genetic engineering techniques to introduce normal genes or therapeutic genes into the cells of a patient, thereby correcting or compensating for genetic defects of the patient, or suppressing the expression of abnormally expressed genes. As used herein, the term “cell therapy” refers to the treatment of diseases by using cells derived from autologous, allogeneic, or xenogeneic sources that have been altered (e.g., genetic engineering) and/or cultured and proliferated ex vivo for therapeutic purposes. Gene therapy or cell therapy may target various genetic and/or acquired diseases, including cancers, genetic disorders, immune disorders, hematologic disorders, neurological disorders, metabolic disorders, cardiovascular diseases, infectious diseases, and organ transplantation.

In the present invention, a representative example of the target gene may be a cellular reprogramming gene. Accordingly, the vector comprising the Lgr4 gene promoter or a fragment thereof, and cell reprogramming genes, when introduced into cells, can induce cell reprogramming through gene expression upon the application of electromagnetic waves, while blocking the electromagnetic waves can reduce or stop cell reprogramming, thereby enabling its application in gene therapy or cell therapy.

As used herein, the term “reprogramming” is used interchangeably with “cell fate conversion” and refers to a method of transforming a specific cell into a desired cell type by regulating the global gene expression pattern and the like. In other words, reprogramming in the present invention refers to a method of artificially manipulating the fate of a cell to convert it into a cell with entirely different characteristics, and for the purpose of the present invention, the reprogramming may be achieved by introducing a vector comprising exogenous genes or other genes into the cell. The reprogramming may refer to cell differentiation, dedifferentiation, direct reprogramming (also referred to as direct conversion), or direct trans-differentiation.

As used herein, the term “direct reprogramming” refers to a technique that induces the conversion between adult cells of entirely different cell types. Direct programming differs from conventional techniques in that the technique induces the direct conversion of adult cells into the desired type of cell without generating induced pluripotent stem cells. In the present invention, the target cells are directly produced from precursor cells through direct reprogramming technology without the generation of induced pluripotent stem cells, offering advantages in terms of production time, cost, efficiency, and safety. As used herein, the term “direct reprogramming” may be interchangeably used with “direct dedifferentiation”, “direct differentiation”, “direct conversion”, “direct trans-differentiation”, “trans-differentiation”, etc. The direct reprogramming may particularly refer to the conversion into neurons or bone cells.

In one embodiment, the cellular reprogramming genes may be genes that reprogram somatic cells into induced pluripotent stem cells through dedifferentiation. For example, the cellular reprogramming genes may be one or more selected from the Oct4 family, Nanog, Sox2, Myc, Klf family (including KLF1, KLF2, KLF3, KLF4, KLF5, KLF6, KLF7, KLF8, KLF9, KLF10, KLF11, KLF12, KLF13, KLF14, KLF15, KLF16, and KLF17), and Lin-28, and specifically one or more selected from Oct4 (octamer-binding transcription factor 4), Nanog (Nanog homeobox), Sox2 (sex determining region Y-box), c-Myc (cellular myelocytomatosis oncogene), Klf4 (Krueppel-like factor 4), Lin28 (Lin-28), and L-Myc. As used herein, a vector comprising the Lgr4 gene promoter or a fragment thereof, and at least one gene selected from Oct4, Nanog, Sox2, c-Myc, Klf4, Lin28, and L-myc, when introduced into cells, can induce reprogramming of somatic cells into induced pluripotent stem cells through dedifferentiation by the application of electromagnetic fields and reduce or cease the reprogramming by the blockage of electromagnetic fields. Accordingly, the vector may be utilized in gene therapy or cell therapy.

Oct4, Sox2, Klf4, and c-Myc genes are known to reprogram somatic cells and induce pluripotency when introduced into somatic cells (Takahashi K, et al., Cell, 126 (4):663-676, 2006; Takahashi K, et al., Cell, 131(5):861-872, 2007). Additionally, Oct4, Sox2, Nanog, and LIN28 genes are also known to reprogram somatic cells and induce pluripotency when introduced into somatic cells (Junying Yu et al., Science, 318 (5858):1917-1920, 2007). It is obvious to a person of ordinary skill in the art that part or all of Oct4, Nanog, Sox2, Klf4, LIN28, and Myc may be substituted with their known functional equivalents.

As used herein, the term “somatic cell” refers to any type of cell that constitutes the body of an organism, excluding germ cells and undifferentiated stem cells. Somatic cells may include, for example, cells of the skin, heart, muscle, nerves, bones, fat, gastrointestinal tract, bone marrow, pancreas, or blood. Somatic cells may be derived from mammals and may be autologous, but are not limited thereto.

As used herein, the term “induced pluripotent stem cell” refers to a cell induced to acquire pluripotency through an artificial dedifferentiation process of differentiated cells and is also referred to as a dedifferentiated induced pluripotent stem cell. Induced pluripotent stem cells exhibit characteristics nearly identical to those of embryonic stem cells, specifically showing similarities in cell morphology, gene and protein expression patterns. The induced pluripotent stem cells possess pluripotency both in vitro and in vivo, form teratomas, and is capable of germline transmission of genes.

In another embodiment, the cellular reprogramming genes may be genes that directly trans-differentiate somatic cells into neurons. For example, the cellular reprogramming genes may be one selected from achaete-scute complex 1 (Ascl1), nuclear receptor related 1 protein (Nurr1), paired-like homeodomain 3 (Pitx3), and LIM homeobox transcription factor 1 alpha (Lmx1a). As used herein, a vector comprising the Lgr4 gene promoter or a fragment thereof, and at least one gene selected from Ascl1, Nurr1, Pitx3, and Lmx1a, when introduced into cells, can induce reprogramming of somatic cells into neurons through differentiation by the application of electromagnetic fields and reduce or cease the reprogramming by the blockage of electromagnetic fields. Accordingly, the vector may be utilized in gene therapy or cell therapy.

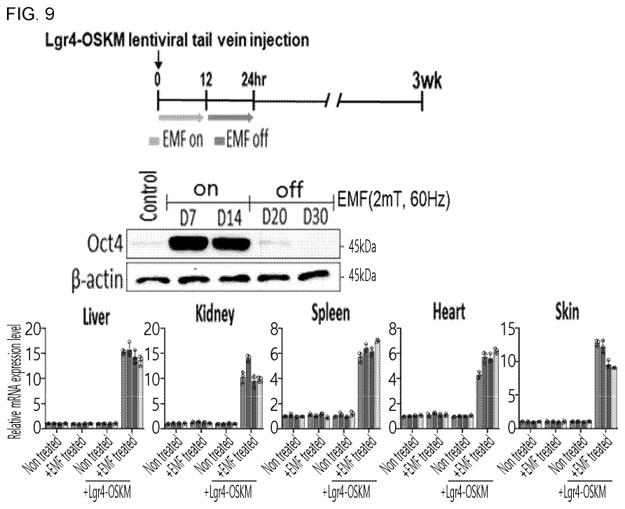

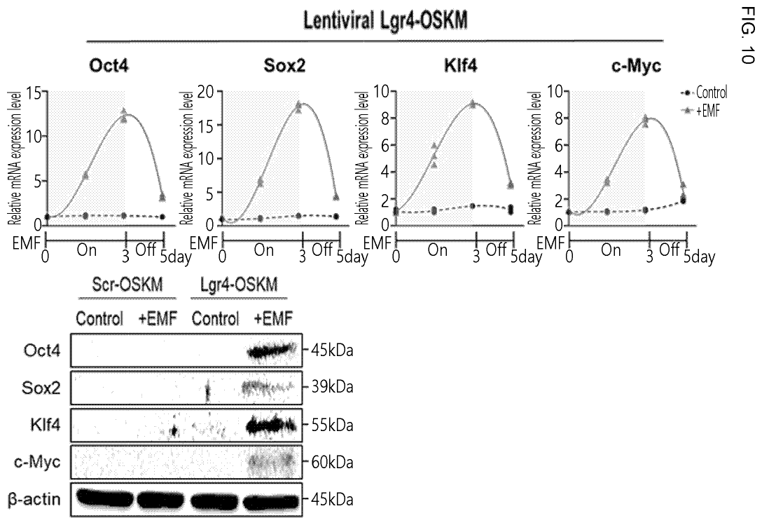

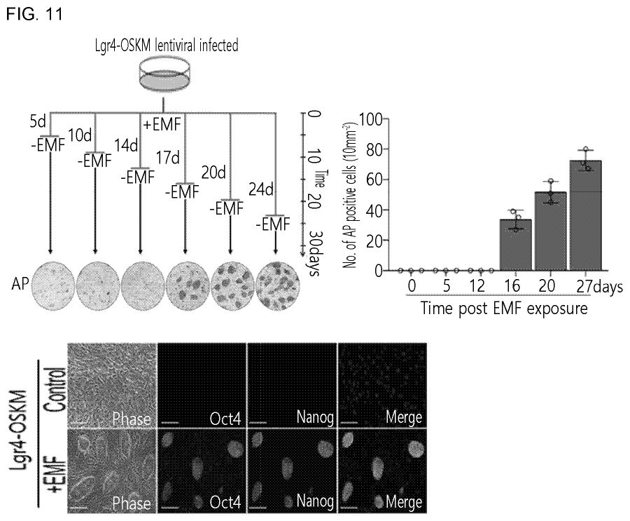

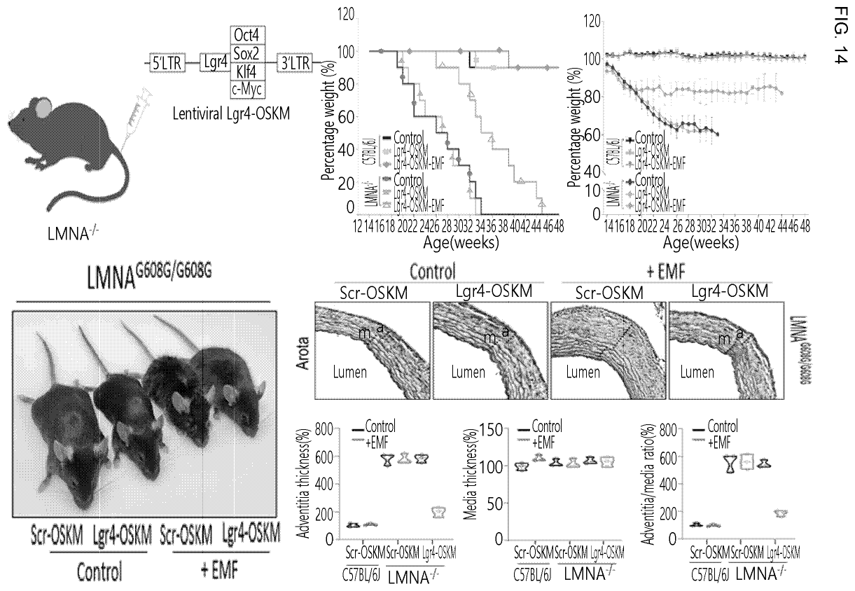

In one embodiment of the present invention, vectors were constructed by operably linking each target gene, which are Oct4, Nanog, Sox2, c-Myc, and Klf4, to the Lgr4 gene promoter. When these vectors were introduced into cells and exposed to electromagnetic fields, the successful generation of induced pluripotent stem cells was confirmed (FIG. 11). Furthermore, when the vector was injected into an aged mouse model, it was confirmed that the genes were expressed during the period of electromagnetic field exposure and not expressed after the blockage of electromagnetic fields (FIG. 9). Additionally, in the aged mouse model, gene expression was confirmed through lifespan extension, increased weight gain, reduced dorsal kyphosis, and amelioration of cardiovascular disease symptoms, a representative sign of aging (FIG. 14).

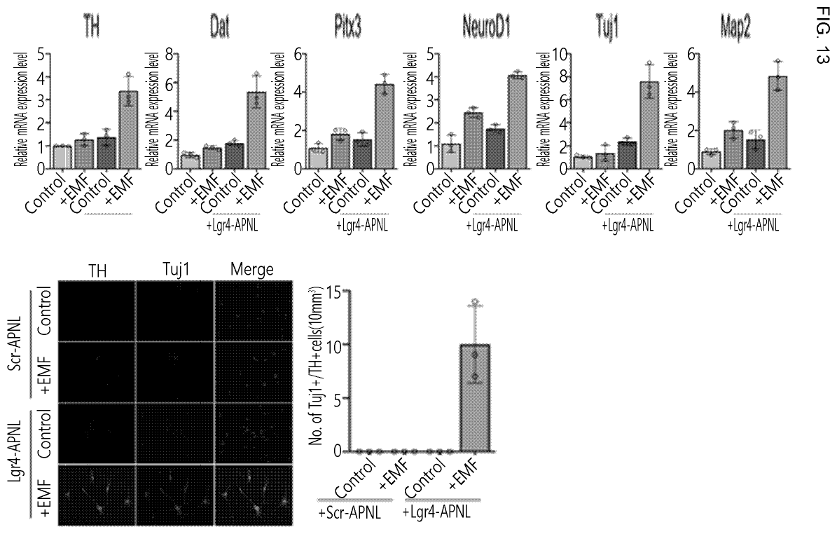

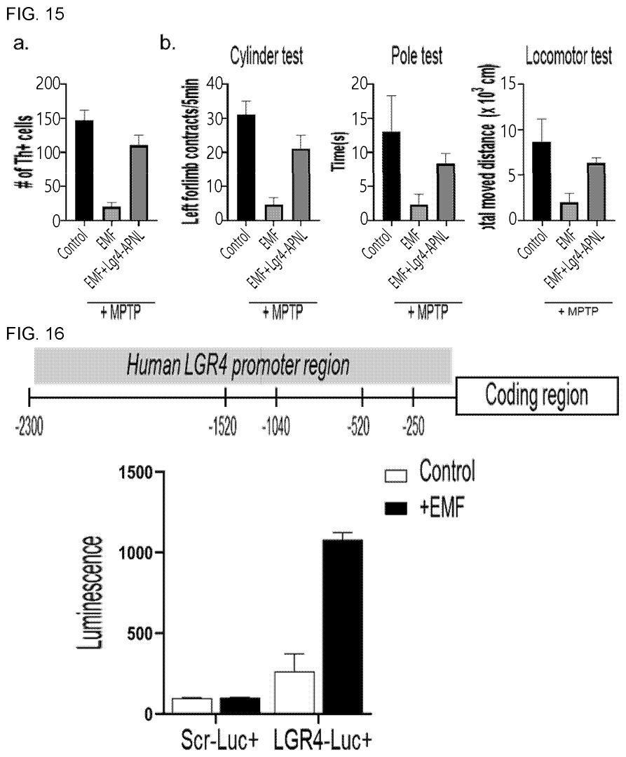

In another embodiment of the present invention, vectors were constructed by operably linking each of the target genes, Ascl1, Nurr1, Pitx3, and Lmx1a, to the Lgr4 gene promoter. When these vectors were introduced into cells and exposed to electromagnetic fields, the successful generation of induced directly trans-differentiated neurons was confirmed (FIG. 13). Furthermore, after injecting vectors comprising the Lgr4 promoter and each of the Ascl1, Pitx3, Nurr1, and Lmx1a (APNL) genes into a Parkinson's disease mouse model, the induced directly transdifferentiated dopaminergic neuronal cells were identified, and behavioral recovery was also observed (FIG. 15).

Accordingly, an embodiment of the present invention provides a vector comprising an Lgr4 gene promoter or a fragment thereof, and a cellular reprogramming gene operably linked thereto.

Additionally, the present invention provides a use of the vector for cellular reprogramming by electromagnetic fields. Specifically, the present invention provides a composition for cellular reprogramming by electromagnetic fields, comprising the vector. Further, the present invention provides a method for cellular reprogramming, comprising: introducing the vector into a cell; and applying electromagnetic fields to the cell or blocking electromagnetic fields from acting on the cell.

Such compositions and methods can regulate cell reprogramming in a manner where the application of electromagnetic fields induces gene expression, thereby resulting in reprogramming, and the blocking of electromagnetic fields reduces or halts gene expression, thereby reducing or ceasing reprogramming.

As a specific example of the reprogramming composition of the present invention, the reprogramming composition may be used for the manufacture of a pharmaceutical composition for the prevention and treatment of degenerative diseases. For example, the pharmaceutical composition may comprise a vector including the promoter of the Lgr4 gene or a fragment thereof and a cell reprogramming gene operably linked thereto, or a cell prepared through the reprogramming method provided in the present invention, as an active ingredient.

Another aspect of the present invention provides a cell therapeutic composition comprising, as an active ingredient, cells produced using the reprogramming method employing the vector described herein.

Another aspect of the present invention provides a pharmaceutical composition for the prevention and treatment of degenerative diseases, which comprises a vector including the promoter of the Lgr4 gene or a fragment thereof operably linked to a cell reprogramming gene, or cells produced using the reprogramming method employing the vector, as an active ingredient.

For example, the degenerative disease may be a neurodegenerative disease.

The neurodegenerative disease may be caused by deformation, loss, or functional decline of neurons or nervous tissues and may include, for example, diseases selected from Parkinson's disease, Alzheimer's disease, Pick's disease, Huntington's disease, amyotrophic lateral sclerosis, ischemic brain diseases (strokes), demyelinating diseases, multiple sclerosis, epilepsy, and spinal cord injury.

### Mode for Carrying Out the Invention

Hereinafter, the present invention will be described in detail by the following Examples. However, these Examples are only for illustrating the present invention, and the scope of the present invention is not limited to these Examples. It is obvious to a person of ordinary skill in the art that the Examples described below may be altered within the scope of the essential spirit of the present invention.

### Example 1. Discovery of Electromagnetic Field-Responsive Promoters

In order to identify the genes whose expression levels change in response to electromagnetic field (EMF) exposure in tissues of various organs, C57BL/6J mice were treated with electromagnetic fields with an intensity of 20 G and at a frequency of 60 Hz for 12 hours per day over 7 days, and tissues were collected from the brains, hippocampi, cortices, hearts, livers, spleens, kidneys, skins, muscles, and lungs of the mice. In each tissue, the gene mRNA expression levels of the group with EMF exposure relative to the mRNA expression levels of the group without EMF exposure (control group) were measured using qRT-PCR. Specifically, cDNA was synthesized using AccuPower RT-PCR PreMix (Bioneer) for qRT-PCR, and qRT-PCR was performed using SYBR Green Real-time PCR Master Mix (Invitrogen). After a 1/50 dilution of the reverse transcription reaction, qRT-PCR analysis was conducted using a Rotor-Gene Q RT-PCR cycler (QIAGEN).

As a result, it was confirmed that the mRNA expression levels of genes such as Lgr4, Dhcr7, Egr1, Insig1, and Olfml3 change in response to EMF. Among these genes, the expression level of the Lgr4 gene responded to EMF in all tested tissues and exhibited significantly superior responsiveness compared to the group without EMF exposure (control group) (see the upper drawing in FIG. 1). Additionally, the Lgr4 gene responded well to EMF with an intensity of approximately 20 G (i.e., 2×10⁻³ T) and a frequency range of approximately 60 Hz to 100 Hz (see the lower drawing in FIG. 1).

### Example 2. Preparation of Vectors Comprising Electromagnetic Field-Responsive Promoters

To verify whether the promoter of the Lgr4 gene responds to EMF exposure, a lentiviral vector was constructed comprising the Lgr4 gene promoter or a fragment thereof (referred to as the ‘EMF response promoter’, ‘ERP’, or ‘E4’), and a luciferase gene or GFP gene (FIG. 2). Specifically, a luciferase plasmid (pGL3-Lgr4) was constructed by inserting the Lgr4 gene promoter (SEQ ID NO: 1) into a pGL3-basic plasmid through Kpn1 and Xho1 restriction enzymes. Additionally, a control plasmid (pGL3-Scr) was constructed using a scrambled sequence that is unrelated to the gene transcription process and not part of the promoter region. Furthermore, to analyze the responsiveness of different regions of the Lgr4 promoter, various regions of the Lgr4 promoter containing elements were constructed. Among these regions, the region closest to ATG (corresponding to the sequence from position 1337 to position 1978 of SEQ ID NO: 1 and presented as SEQ ID NO: 3) was named E4. The following primers were used for cloning the Lgr4 promoter:

Lgr4 promoter (full length) forward:
(SEQ ID NO: 4)
5′ GGCAGAGGCAGGCAAATTTC 3′,
Lgr4 promoter (full length) reverse:
(SEQ ID NO: 5)
5′ CTCGCTTCCTTCAGCAGTCT 3′,
Lgr4 promoter (E4) forward:
(SEQ ID NO: 6)
5′ GGAGGGAAACAGCAGACTCC 3′,
Lgr4 promoter (E4) reverse:
(SEQ ID NO: 7)
5′ CTCGCTTCCTTCAGCAGTCT 3′.

As a result, it was confirmed that the plasmid comprising E4 (pGL3-Lgr4-E4), the region closest to ATG, exhibited the highest responsiveness to EMF treatment compared to leaky expression, which was greater than that of the plasmid comprising the full Lgr4 sequence (pGL3-Lgr4-FL) (FIG. 3a). Accordingly, E4 was used as the EMF response promoter (ERP).

The plasmid comprising the GFP gene was cloned using pCIG3 (CMV-IRES-EGFP). The CMV promoter between the 5′-LTR and 3′-LTR was removed using Spe1 and EcoRV restriction enzymes, and ligation was performed by introducing the Lgr4 promoter. Then, the insertion of the Lgr4 promoter at the 5′ end of the GFP gene was confirmed by Sanger sequencing. The GFP gene was already inserted into the original plasmid.

### Example 3. Confirmation of Regulation of Target Gene Expression Using Electromagnetic Field-Responsive Promoters

### 3-1. Luminescence Assay Using Luciferase in Cells

The vector prepared in Example 2, which includes the luciferase gene as the target gene, was introduced into fibroblasts, 3T3 cells, neurons, astrocytes, and cardiomyocytes. The level of luminescence resulting from luciferase expression was confirmed using a luminometer (Lubi) and the Lubi2 program, based on the luminescence assay system from Promega. The results are shown in FIG. 3. A scrambled promoter was used as the control.

FIG. 3a shows the results of introducing pGL3-basic, pGL3-scr, and pGL3-Lgr4-E4 into mouse embryonic fibroblast cells and applying (on) or blocking (off) electromagnetic fields (20 G and 60 Hz). It was confirmed that when the Lgr4 promoter was introduced, luminescence increased upon EMF application due to the induction of luciferase gene expression, and luminescence disappeared upon blockage of EMF due to the cessation of luciferase gene expression.

FIG. 3b shows the results of introducing pGL3-Lgr4-E4 into mouse fibroblast cells and applying (on) or blocking (off) electromagnetic fields under various conditions (0 mT to 10 mT, and 0 Hz to 150 Hz). It was confirmed that the Lgr4 promoter showed the highest sensitivity to EMF under conditions of approximately 20 G to 30 G and 60 Hz to 150 Hz.

FIG. 3c shows the results of introducing pGL3-scr and pGL3-Lgr4-E4 into fibroblasts, 3T3 cells, neurons, astrocytes, and cardiomyocytes, and applying (on) or blocking (off) EMF (20 G and 60 Hz). It was confirmed that the Lgr4 promoter responded sensitively to EMF in all tested cell types.

### 3-2. Fluorescence Analysis Using GFP in Cells

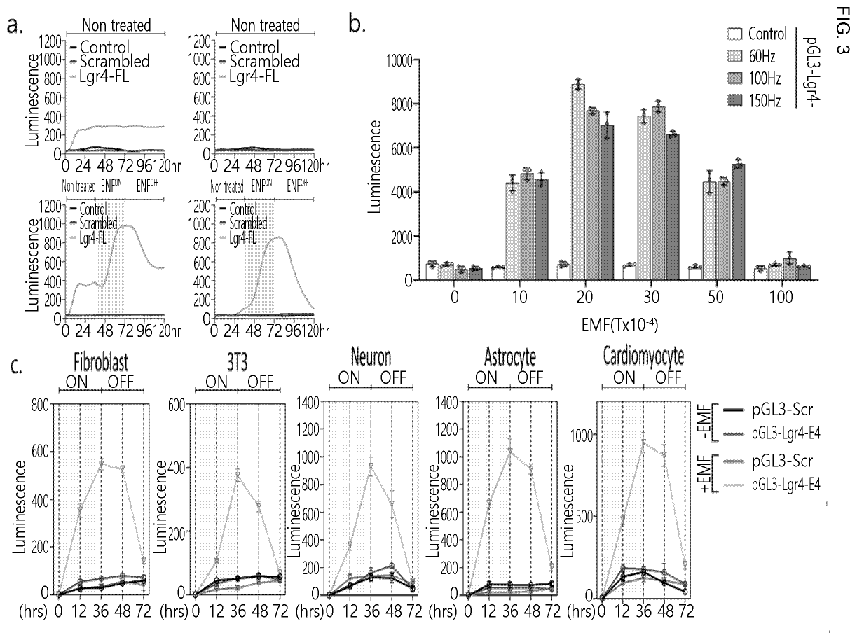

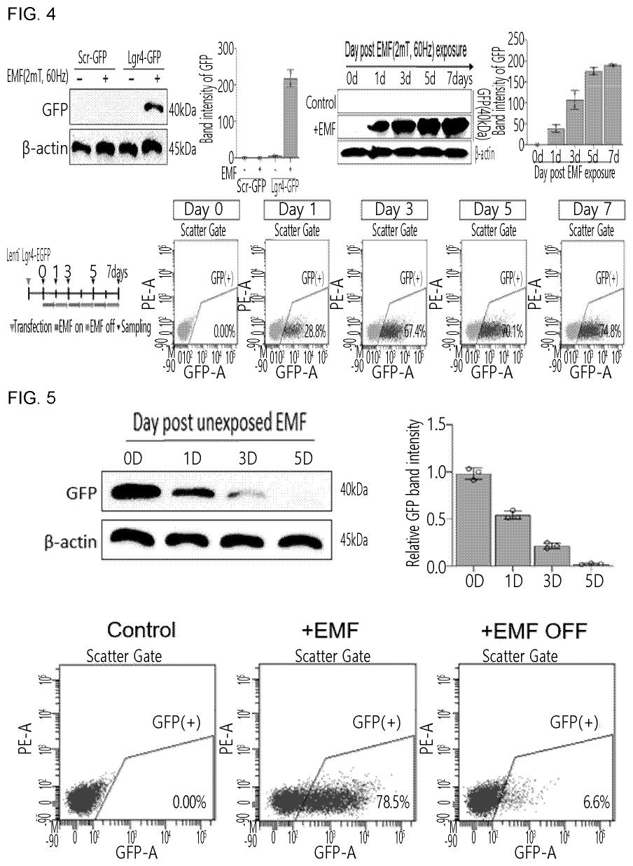

The vector prepared in Example 2, which includes the GFP gene as the target gene, was introduced into mouse fibroblast cells, and EMF (20 G and 60 Hz) was applied (on) or blocked (off). The expression level of the GFP gene was confirmed by Western blotting and FACS, and the results are shown in FIG. 4 and FIG. 5.

For Western blotting, the proteins of Lgr4-GFP virus-infected cells were extracted from a lysis buffer, containing 1% NP-40, 0.5% deoxycholate (DOC), 0.1% SDS, 150 mmol/L NaCl, and 50 mmol/L Tris (pH 8.0) (Sigma-Aldrich), along with a 1× proteinase inhibitor mixture (Roche), using 1× phosphate-buffered saline (PBS). The extracted proteins were separated by 12% sodium dodecyl sulfate-polyacrylamide gel electrophoresis (SDS-PAGE) and then transferred onto a nitrocellulose membrane. This membrane contains GFP (1:1000, Abcam) and beta-actin (1:1000, AbFrontier). Representative images of the Western blot were visualized using the Chemidoc TRS+ with Image Lab software (Bio-Rad).

The GFP expression level induced by the EMF was measured using flow cytometry (FACS). Specifically, cells were dissociated using trypsin for 5 minutes, followed by pelleting of the single cells and resuspending the single cells in ice-cold 4% paraformaldehyde, then incubating at 4° C. for 10 minutes. The cells were washed twice and resuspended in FACS buffer for FACS analysis. Using a 488 nm laser, the cell population separated in the FL1 channel was analyzed for GFP-positive cells. A scrambled promoter was used as the control.

FIG. 4 shows that minimal GFP expression was observed in the control group regardless of EMF exposure, whereas in the experimental group using the Lgr4 promoter, GFP was expressed upon EMF application, with the expression increasing over time, and was absent or reduced when EMF was blocked.

FIG. 5 shows the results of verifying GFP expression using Western blotting and FACS after the discontinuance of EMF exposure. It was confirmed that when the EMF exposure was discontinued, GFP expression in the experimental group using the Lgr4 promoter ceased, resulting in a decrease in GFP expression over time, and consequently, the gradual disappearance of the fluorescent protein. These results indicate that the Lgr4 promoter can be selectively activated only when EMF is applied.

### 3-3. Fluorescence Analysis Using GFP in Animal Models

Using the electromagnetic field-responsive gene (Lgr4) promoter prepared in Example 2, a gene fragment containing the GFP gene as a target gene was injected into a mouse at the one-cell stage, and the Lgr4 gene promoter electromagnetic field-responsive gene was randomly introduced into the mouse genome, thereby generating a transgenic mouse model. EMF (20 G, 60 Hz) was applied (on) or blocked (off) to this mouse model for 12 hours per day over five days, and in vivo GFP expression in various organs was measured using Western blotting and immunostaining. DAPI staining was performed as a counterstain. Specifically, each organ was dissociated using a homogenizer, and proteins were extracted from the lysis buffer containing 1× phosphate-buffered saline (PBS), 1% NP-40, 0.5% deoxycholate (DOC), 0.1% SDS, 150 mmol/L NaCl, 50 mmol/L Tris (pH 8.0, Sigma-Aldrich), using a 1× protease inhibitor mixture (Roche). The extracted proteins were separated by 12% sodium dodecyl sulfate-polyacrylamide gel electrophoresis (SDS-PAGE) and then transferred onto a nitrocellulose membrane. This membrane contains GFP (1:1000, Abcam) and beta-actin (1:1000, AbFrontier). Representative images of the Western blot were visualized using the Chemidoc TRS+ with Image Lab software (Bio-Rad). Additionally, for the immunostaining experiment, in vivo organs were fixed onto blocks using paraffin, and then micro sections of the organs were placed onto slide glasses using a microtome. After removing paraffin using a standardized deparaffinization protocol, immunostaining was performed according to standard procedures using the following primary antibodies: GFP (Abcam) and fluorescent secondary antibodies (Invitrogen).

The results are shown in FIG. 6, and it was confirmed that the Lgr4 promoter was activated in most organs in vivo of the transgenic mouse upon EMF exposure.

### Example 4. Comparison of Electromagnetic Field-Responsiveness of Electromagnetic Field-Responsive Promoters

To compare the electromagnetic field responsiveness of Egr1 gene promoter and Ifi44 promoter, which are commonly known electromagnetic field-responsive promoters, and the Lgr4 gene promoter according to the present invention, lentiviral vectors with GFP genes ligated to each promoter were generated and introduced into mouse fibroblast cells using the method described in Example 2, and following EMF exposure, GFP expression was confirmed through fluorescence microscopy and immunostaining. Immunocytochemistry was performed by fixing the cells in PBS using 4% paraformaldehyde, followed by immunostaining according to standard protocols using the following primary antibodies: GFP (Abcam) and fluorescent secondary antibodies (Invitrogen). Additionally, GFP expression induced by the electromagnetic fields was measured using flow cytometry (FACS). All flow cytometry analyses were conducted using an Accuri device (Becton-Dickinson). Data were analyzed using FlowJo software (TreeStar). Specifically, cells were dissociated using trypsin for 5 minutes, followed by pelleting of the single cells and resuspending the single cells in ice-cold 4% paraformaldehyde, then incubating at 4° C. for 10 minutes. The cells were washed twice and resuspended in FACS buffer for FACS analysis.

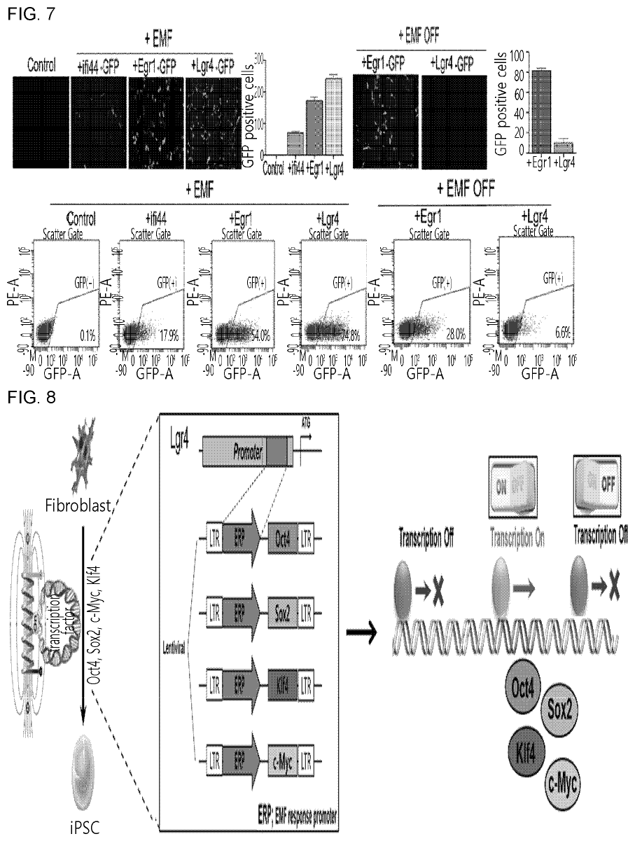

The results are shown in FIG. 7. Specifically, the immunostaining results indicated that, upon electromagnetic field exposure (on), the responsiveness was superior in the following order: Ifi44 gene promoter, Egr1 gene promoter, and Lgr4 gene promoter. Additionally, when the EMF was blocked (off), it was observed that the number of GFP-expressing cells decreased to the control level for the Lgr4 gene promoter, whereas, the number of GFP-expressing cells did not decrease for the Egr1 promoter, even after the EMF was blocked (off). Additionally, FACS results also confirmed that the Lgr4 gene promoter exhibited more than four times and 1.5 times higher responsiveness to EMF compared to the Egr1 and Ifi44 gene promoters, respectively. When the EMF was blocked (off), it was found that the Lgr4 gene promoter was over four times more effective compared to the Egr1 gene promoter.

### Example 5. Preparation of Induced Pluripotent Stem Cells Using Electromagnetic Field-Responsive Promoters

In order to induce dedifferentiation reprogramming of somatic cells into induced pluripotent stem cells, the genes to be overexpressed, such as Oct4, Nanog, Sox2, c-Myc, and Klf4, were set as target genes, and lentiviral vectors were constructed with the Lgr4 gene promoter or a fragment thereof (i.e., “EMF response promoter (ERP)”) and each of the target genes, Oct4, Nanog, Sox2, c-Myc, or Klf4. FIG. 8 provides a schematic diagram and an overview of the operation of each vector. The method for constructing the vectors was essentially the same as described in Example 2, except that to clone each target gene, Asc1 and EcoRV restriction enzymes were used to replace the eGFP sequence with the protein coding regions of the Oct4, Sox2, c-Myc, and Klf4 genes by ligation. The primers for cloning the coding regions of the Oct4, Sox2, c-Myc, and Klf4 genes, as well as the primers for cloning the 4F2A coding gene, are as follows:

Oct4 cds forward:
(SEQ ID NO: 8)
5′ ATGGCTGGACACCTGGCTTC 3′,
Oct4 cds reverse:
(SEQ ID NO: 9)
5′ TCAGTTTGAATGCATGGGAG 3′,
Sox2 cds forward:
(SEQ ID NO: 10)
5′ ATGTATAACATGATGGAGAC 3′,
Sox2 cds reverse:
(SEQ ID NO: 11)
5′ TGCCCCTGTCGCACATGTGA 3′,
c-Myc cds forward:
(SEQ ID NO: 12)
5′ ATGCCCCTCAACGTGAACTT 3′,
c-Myc cds reverse:
(SEQ ID NO: 13)
5′ TTATGCACCAGAGTTTCGAAGCTG 3′,
Klf4 cds forward:
(SEQ ID NO: 14)
5′ ATGAGGCAGCCACCTGGCGA 3′,
Klf4 cds reverse:
(SEQ ID NO: 15)
5′ TTAAAAGTGCCTCTTCATGTGT 3′

After introducing the constructed vectors into somatic cells, the expression of each target gene was confirmed using qRT-PCR and Western blotting (FIG. 10).

Additionally, EMF exposure at 20 G and 60 Hz was applied to the cells introduced with the constructed vectors. The successful generation of induced pluripotent stem cells was confirmed by analyzing the number of alkaline phosphatase-positive cells (a marker for pluripotency) and through immunostaining for the Nanog and Oct4 genes (FIG. 11).

### Example 6. Direct Trans-Differentiation of Neurons Using Electromagnetic Field-Responsive Promoters

To directly transdifferentiate somatic cells into neurons, the genes to be expressed, including Ascl1, Nurr1, Pitx3, and Lmx1a genes are set as the target genes, and lentiviral vectors were constructed with the Lgr4 gene promoter or a fragment thereof (i.e., “EMF response promoter (ERP)”), and each of the target genes Ascl1, Nurr1, Pitx3, or Lmx1a. The method for constructing the vectors was essentially the same as described in Example 2, except that each gene was cloned into the Lgr4-GFP vector via ligation, using EcoRI restriction enzyme to extract the genes from the FUW-ANPL vector. After introducing the prepared vectors into somatic cells, qRT-PCR was performed using the primers Ascl1, Pitx3, Nurr1, and Lmx1a following the same protocols as the original method. The primers used for qRT-PCR are as follows:

Ascl1 forward:
(SEQ ID NO: 16)
5′ TCCAGGGTTTAGGGTTGGGA 3′
Ascl1 reverse:
(SEQ ID NO: 17)
5′ CCTTCCTACAAACGCCTCGT 3′,
Pitx3 forward:
(SEQ ID NO: 18)
5′ TTTCGCAACGGGTTTGCCGC 3′,
Pitx3 reverse:
(SEQ ID NO: 19)
5′ AAGGTCGCCTCTAGCTCCTGTAG 3′,
Nurr1 forward:
(SEQ ID NO: 20)
5′ CTCCCTCCATGAGGGTCTG 3′,
VNurr1 reverse:
(SEQ ID NO: 21)
5′ TCTTCGGCTTCGAGGGTAAA 3′,
Lmx1a forward:
(SEQ ID NO: 22)
5′ GCAAAGGGGACTATGAGAAGGA 3′,
Lmx1a reverse:
(SEQ ID NO: 23)
5′ CGTTTGGGGCGCTTATGGT 3′.

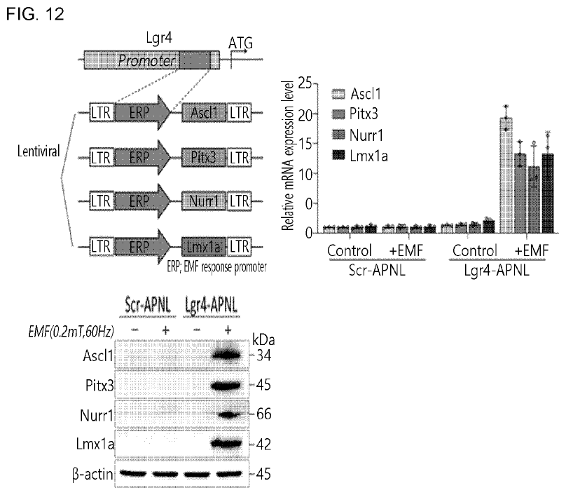

The expression of each target gene was confirmed through Western blotting using primary antibodies for Ascl (abcam), Pitx3 (abcam), Nurr1 (abcam), and Lmx1a (abcam). FIG. 12 shows a schematic diagram of each vector and the results of verifying the expression of each gene following electromagnetic field (EMF) treatment.

Additionally, after applying EMF at 20 G and 60 Hz to the cells introduced with the constructed vectors, the expression of neuronal markers, including TH, Dat, Pitx3, NeuroD1, Tuj1, and Map2 genes, was measured using qRT-PCR. Additionally, immunostaining for TH and Tuj1 genes confirmed that induced directly transdifferentiated neurons were generated only in the experimental group exposed to EMF (FIG. 13).

### Example 7. Treatment of Aging through Transient Gene Expression Induced by EMF

A vector containing the Lgr4 promoter and the genes Oct4, Nanog, Sox2, c-Myc, and Klf4 (collectively known as OSKM), as prepared in Example 5, was injected into aged mouse models (C57BL/6-Tg (LMNA*G608G) HCIns/J, purchased from Jackson Laboratory) via the tail vein. The mice were exposed to an EMF at 20 G and 60 Hz for 12 hours per day for 14 days, after which the EMF was blocked (off). Western blotting and qRT-PCR results confirmed that the OSKM genes were expressed in all organs during EMF treatment. In particular, it was observed that the Oct4 gene was transiently expressed in the mouse heart during the EMF treatment but was not expressed after the EMF was blocked (FIG. 9).

Furthermore, the experimental group where a vector containing the Lgr4 promoter and the genes Oct4, Nanog, Sox2, c-Myc, and Klf4 in a polycistronic form was introduced into aged mouse models, and the mice were applied with a transient EMF exposure (20 G and 60 Hz). The experimental group demonstrated improvements, including increased lifespan, higher weight gain rate, reduced dorsal kyphosis, and improvement in cardiovascular conditions, which are typical signs of aging, compared to the control group (FIG. 14). Additionally, 12 mice were used per experimental group for the lifespan extension and weight measurement experiments. Cardiovascular disease, a characteristic symptom of the aged mouse model, was analyzed by measuring the ratio of the adventitia to the media of the mouse artery. The artery was processed into paraffin blocks, followed by standardized Hematoxylin and Eosin (H&E) staining. Five mice were used per experimental group for the measurements of cardiovascular disease.

### Example 8. Treatment of Parkinson's Disease Through Transient Gene Expression Induced by EMF

A vector containing the Lgr4 promoter and the genes Ascl1, Pitx3, Nurr1, and Lmx1a (collectively known as “APNL”), as prepared in Example 5, was injected into the striatum of mouse models in which Parkinson's disease had been induced with 1-methyl-4-phenyl-1,2,3,6-tetrahydropyridine (MPTP). The mice were then exposed to an EMF at 20 G and 60 Hz for 12 hours per day for 14 days. The results confirmed by immunostaining showed that the number of dopamine neuron-positive cells increased under the condition where both the EMF and the Lgr4-APNL vector were applied together. Additionally, behavioral experiments confirmed that mice treated with both the EMF and the Lgr4-APNL vector showed significant behavioral improvement compared to the mouse models with Parkinson's disease that are treated with MPTP alone (FIG. 15).

### Example 9. Confirmation of Target Reporter Gene Responsiveness Using Electromagnetic Field-Responsive Human Promoters

To verify whether the promoter of the human Lgr4 gene responds to EMF exposure, a lentiviral vector was constructed comprising the human Lgr4 gene promoter and a luciferase gene (FIG. 16). Specifically, a luciferase plasmid (pGL3-Lgr4) was constructed by inserting the human Lgr4 gene promoter into a pGL3-basic plasmid through Kpn1 and Xho1 restriction enzymes. The sequences of the primers used are as follows:

Lgr4 Promoter Forward:
(SEQ ID NO: 24)
GTAGAACCGAAGTCCAGCTTATC
Lgr4 Promoter Reverse:
(SEQ ID NO: 25)
GGAGGTCTCGAGCTCATTACTA

Additionally, a control plasmid (pGL3-Scr) was constructed using a scrambled (Scr) sequence that is unrelated to the gene transcription process and not part of the promoter region. After transducing human fibroblasts with a plasmid containing the Lgr4 gene promoter and luciferase, luciferase activity was assessed three days later, and it was confirmed that the plasmid responded to the EMF (FIG. 16).

As set forth above a person skilled in the art will be able to understand that the present invention may be embodied in other specific forms without departing from the technical spirit or essential characteristics thereof. Accordingly, the embodiments described above should be construed as being exemplified and not limiting the present disclosure. The scope of the present invention should be understood to include all changes or modifications derived from the definitions and scopes of the claims and their equivalents, rather than from the detailed description.

1. (canceled)

2. A vector comprising a nucleic acid having promoter activity regulated by electromagnetic fields, comprising a promoter sequence of Lgr4 gene or a fragment thereof.

3. The vector of claim 2, wherein the promoter of the Lgr4 gene is represented by the nucleotide sequence of SEQ ID NO: 1 or SEQ ID NO: 2, and wherein the fragment thereof is a fragment comprising the nucleotides from position 1337th to position 1978th of SEQ ID NO: 1 or a fragment of SEQ ID NO: 2 corresponding thereto.

4. The vector of claim 2, further comprising a target gene that is operably linked to the nucleic acid.

5. A composition for regulating gene expression by electromagnetic fields, comprising the vector according to claim 2.

6. The composition of claim 5, wherein the application of electromagnetic fields induces the expression of a gene and the blockage of electromagnetic fields reduces the expression of a gene.

7. The composition of claim 6, wherein the electromagnetic fields are applied with an intensity of 10 G or more and 30 G or less, or at a frequency of 50 Hz or more and 300 Hz or less.

8. The composition of claim 5, wherein the target gene is a cellular reprogramming gene.

9. The composition of claim 8, wherein the cellular reprogramming gene is one or more selected from the group consisting of Oct4, Nanog, Sox2, c-Myc, Klf4, Lin28, and L-Myc, and wherein the application of electromagnetic fields induces the expression of the genes to promote reprogramming of somatic cells into induced pluripotent stem cells.

10. The composition of claim 8, wherein the cellular reprogramming gene is one or more selected from the group consisting of Ascl1, Nurr1, Pitx3, and Lmx1a, and wherein the application of electromagnetic fields induces the expression of the genes to promote reprogramming of somatic cells into neurons.

11. The composition of claim 5, wherein the composition is for use in gene therapy or cell therapy.

12. A method for regulating gene expression, comprising: introducing the vector according to claim 2 into a cell; and applying electromagnetic fields to the cell or blocking electromagnetic fields from acting on the cell.

13. The method of claim 12, wherein the application of electromagnetic fields induces the expression of a gene and the blockage of electromagnetic fields reduces the expression of a gene.

14. The method of claim 13, wherein the electromagnetic fields are applied with an intensity of 10 G or more and 30 G or less, or at a frequency of 50 Hz or more and 300 Hz or less.

15. The method of claim 12, wherein the gene is a cellular reprogramming gene, and wherein the application of electromagnetic fields induces expression of the gene to promote cell reprogramming.

16. The method of claim 15, wherein the cellular reprogramming gene is one or more selected from the group consisting of Oct4, Nanog, Sox2, c-Myc, Klf4, Lin28, and L-Myc, and wherein the application of electromagnetic fields induces the expression of the genes to promote reprogramming of somatic cells into induced pluripotent stem cells.

17. The method of claim 15, wherein the cellular reprogramming gene is one or more selected from the group consisting of Ascl1, Nurr1, Pitx3, and Lmx1a, and wherein the application of electromagnetic fields induces the expression of the genes to promote reprogramming of somatic cells into neurons.

18. The vector of claim 2, wherein the target gene is a cellular reprogramming gene.

19. (canceled)

20. A method for cellular reprogramming, comprising:
introducing the vector of claim 18 into a cell; and
applying electromagnetic fields to the cell or blocking electromagnetic fields from acting on the cell.

21. A method for preparing cell therapeutic agent, comprising preparing a cell by introducing the vector of claim 18 into a cell; and applying electromagnetic fields to the cell or blocking electromagnetic fields from acting on the cell.
